(19) United States
(12) Patent Application Publication
(10) Pub. No.: US 2024/0215925 A1
(43) Pub. Date: Jul. 4, 2024

(54) METHOD, SYSTEM, AND NON-TRANSITORY COMPUTER-READABLE RECORDING MEDIUM FOR ESTIMATING ARRHYTHMIA USING COMPOSITE ARTIFICIAL NEURAL NETWORK

(71) Applicant: HUINNO, CO., LTD., Seoul (KR)

(72) Inventors: Sung Hoon JUNG, Seoul (KR); Jin Guk KIM, Seoul (KR); Jae Seong JANG, Seoul (KR)

(21) Appl. No.: 18/606,993

(22) Filed: Mar. 15, 2024

Related U.S. Application Data
(63) Continuation of application No. PCT/KR2023/012021, filed on Aug. 14, 2023.

(30) Foreign Application Priority Data
Aug. 30, 2022 (KR) 10-2022-0109500

(51) Int. Cl. A61B 5/00

(52) U.S. Cl. CPC A61B 5/7267

(57) ABSTRACT

A method for estimating arrhythmia using a composite artificial neural network includes the steps of estimating a class corresponding to a beat segment included in a first section of an electrocardiogram (ECG) signal, using a first artificial neural network; estimating a class corresponding to the first section of the ECG signal, using a second artificial neural network; and mutually verifying the estimated class corresponding to the beat segment included in the first section of the ECG signal and the estimated class corresponding to the first section of the ECG signal.

### CROSS-REFERENCE TO RELATED APPLICATION(S)

This application is a Continuation of International Application No. PCT/KR2023/012021 filed on Aug. 14, 2023, which claims priority to Korean Patent Application No. 10-2022-0109500 filed on Aug. 30, 2022. The aforementioned applications are incorporated herein by reference in their entireties.

### TECHNICAL FIELD

The present invention relates to a method, system, and non-transitory computer-readable recording medium for estimating arrhythmia using a composite artificial neural network.

### RELATED ART

Due to recent rapid progress in science and technology, the quality of life of all mankind is being enhanced and medical environment has changed a great deal. Particularly, in recent years, wearable monitoring devices that can analyze electrocardiogram (ECG) signals and estimate arrhythmia during daily life without visiting a hospital have become widely available to the public.

Such a wearable monitoring device is typically provided with an artificial intelligence model to estimate arrhythmia from an ECG signal, and the artificial intelligence model is conventionally implemented on the basis of an artificial neural network trained to estimate which type of arrhythmia a given section of the ECG signal corresponds to.

However, the artificial neural network trained to estimate which type of arrhythmia the given section of the ECG signal corresponds to has a limitation in that it cannot accurately estimate arrhythmia that may be estimated on a beat segment basis (e.g., atrial premature contraction (APC), ventricular premature contraction (VPC), left bundle branch block (LBBB), and right bundle branch block (RBBB)), which causes a problem that a conventional wearable monitoring device is unable to accurately identify the number or proportion in which arrhythmia that may be estimated on a beat segment basis occurs in the given section of the ECG signal.

### SUMMARY

One object of the present invention is to solve all the above-described problems in the prior art.

Another object of the invention is to improve the accuracy of arrhythmia estimation by compositely using an artificial neural network trained to estimate which type of arrhythmia a beat segment included in a given section of an electrocardiogram (ECG) signal corresponds to, and an artificial neural network trained to estimate which type of arrhythmia the given section of the ECG signal corresponds to.

The representative configurations of the invention to achieve the above objects are described below.

According to one aspect of the invention, there is provided a method for estimating arrhythmia using a composite artificial neural network, comprising the steps of: estimating a class corresponding to a beat segment included in a first section of an electrocardiogram (ECG) signal, using a first artificial neural network; estimating a class corresponding to the first section of the ECG signal, using a second artificial neural network; and mutually verifying the estimated class corresponding to the beat segment included in the first section of the ECG signal and the estimated class corresponding to the first section of the ECG signal.

According to another aspect of the invention, there is provided a system for estimating arrhythmia using a composite artificial neural network, comprising: a first estimation unit configured to estimate a class corresponding to a beat segment included in a first section of an electrocardiogram (ECG) signal, using a first artificial neural network; a second estimation unit configured to estimate a class corresponding to the first section of the ECG signal, using a second artificial neural network; and a verification unit configured to mutually verify the estimated class corresponding to the beat segment included in the first section of the ECG signal and the estimated class corresponding to the first section of the ECG signal.

In addition, there are further provided other methods and systems to implement the invention, as well as non-transitory computer-readable recording media having stored thereon computer programs for executing the methods.

According to the invention, it is possible to improve the accuracy of arrhythmia estimation by compositely using an artificial neural network trained to estimate which type of arrhythmia a beat segment included in a given section of an ECG signal corresponds to, and an artificial neural network trained to estimate which type of arrhythmia the given section of the ECG signal corresponds to.

### BRIEF DESCRIPTION OF THE DRAWINGS

FIG. 1 schematically shows the configuration of an entire system for estimating arrhythmia using a composite artificial neural network according to one embodiment of the invention.

FIG. 2 specifically shows the internal configuration of an arrhythmia estimation system according to one embodiment of the invention.

FIG. 3 schematically shows a process of mutual verification according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the invention will be described in detail with reference to the accompanying drawings to enable those skilled in the art to easily implement the invention.

### Configuration of the Entire System

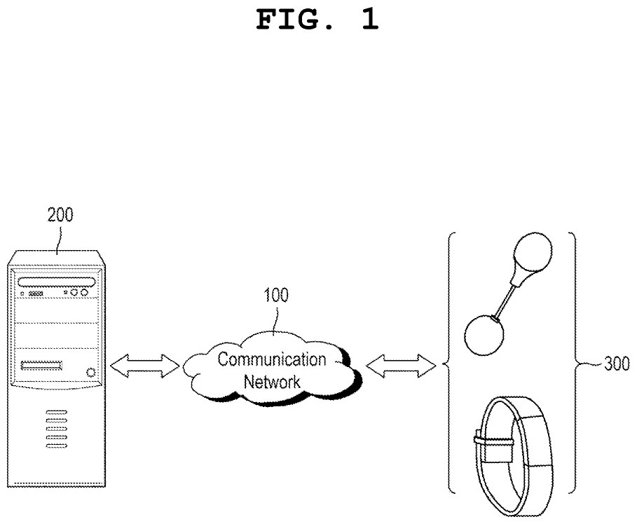

FIG. 1 schematically shows the configuration of the entire system for estimating arrhythmia using a composite artificial neural network according to one embodiment of the invention.

As shown in FIG. 1, the entire system according to one embodiment of the invention may comprise a communication network 100, an arrhythmia estimation system 200, and a device 300.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, 5G communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the arrhythmia estimation system 200 according to one embodiment of the invention may communicate with the device 300 to be described below via the communication network 100. Further, the arrhythmia estimation system 200 according to one embodiment of the invention may function to: estimate a class corresponding to a beat segment included in a first section of an electrocardiogram (ECG) signal, using a first artificial neural network; estimate a class corresponding to the first section of the ECG signal, using a second artificial neural network; and mutually verify the estimated class corresponding to the beat segment included in the first section of the ECG signal and the estimated class corresponding to the first section of the ECG signal. Meanwhile, the arrhythmia estimation system 200 may be digital equipment having a memory means and a microprocessor for computing capabilities, and may be, for example, a server system operating on the communication network 100.

The configuration and functions of the arrhythmia estimation system 200 according to one embodiment of the invention will be discussed in detail below.

Next, the device 300 according to one embodiment of the invention is digital equipment capable of connecting to and then communicating with the arrhythmia estimation system 200, and having a memory means and a microprocessor for computing capabilities, such as a smart patch, a smart watch, a smart band, and smart glasses, and may be a wearable monitoring device including a sensing means (e.g., a contact electrode) for measuring a biosignal (e.g., an ECG signal) from a human body, and a display means for providing a user with a variety of information on the measurement of the biosignal.

Further, according to one embodiment of the invention, the device 300 may further include an application program for performing the functions according to the invention. The application may reside in the device 300 in the form of a program module. The characteristics of the program module may be generally similar to those of a first estimation unit 210, a second estimation unit 220, a verification unit 230, a communication unit 240, and a control unit 250 of the arrhythmia estimation system 200 to be described below. Here, at least a part of the application may be replaced with a hardware device or a firmware device that may perform a substantially equal or equivalent function, as necessary.

### Configuration of the Arrhythmia Estimation System

Hereinafter, the internal configuration of the arrhythmia estimation system 200 crucial for implementing the invention and the functions of the respective components thereof will be discussed.

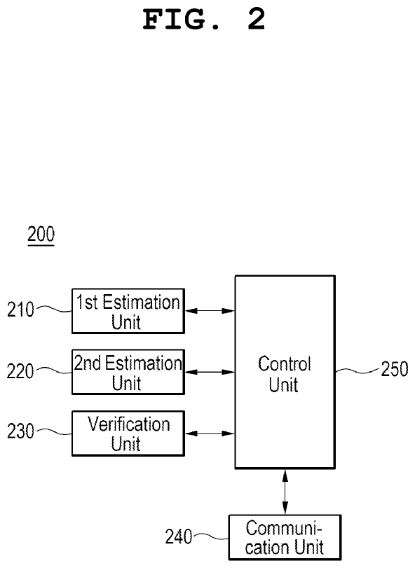

FIG. 2 specifically shows the internal configuration of the arrhythmia estimation system 200 according to one embodiment of the invention.

As shown in FIG. 2, the arrhythmia estimation system 200 according to one embodiment of the invention may comprise a first estimation unit 210, a second estimation unit 220, a verification unit 230, a communication unit 240, and a control unit 250. According to one embodiment of the invention, at least some of the first estimation unit 210, the second estimation unit 220, the verification unit 230, the communication unit 240, and the control unit 250 of the arrhythmia estimation system 200 may be program modules to communicate with an external system (not shown). The program modules may be included in the arrhythmia estimation system 200 in the form of operating systems, application program modules, or other program modules, while they may be physically stored in a variety of commonly known storage devices. Further, the program modules may also be stored in a remote storage device that may communicate with the arrhythmia estimation system 200. Meanwhile, such program modules may include, but are not limited to, routines, subroutines, programs, objects, components, data structures, and the like for performing specific tasks or executing specific abstract data types as will be described below in accordance with the invention.

Meanwhile, the above description is illustrative although the arrhythmia estimation system 200 has been described as above, and it will be apparent to those skilled in the art that at least a part of the components or functions of the arrhythmia estimation system 200 may be implemented in the device 300 or a server (not shown) or included in an external system (not shown), as necessary.

First, the first estimation unit 210 according to one embodiment of the invention may function to estimate a class corresponding to a beat segment included in a first section of an electrocardiogram (ECG) signal, using a first artificial neural network.

Here, the first artificial neural network according to one embodiment of the invention may be an artificial neural network trained to estimate which of classes representing a first type of arrhythmia a beat segment included in a section of the ECG signal corresponds to. Here, the beat segment may refer to a QRS waveform (or QRS complex) appearing in the ECG signal, and may be detected from the ECG signal by the first artificial neural network, or by a means or method other than the first artificial neural network. According to one embodiment of the invention, the first type of arrhythmia may include arrhythmia that may be estimated on a beat segment basis, and may include, for example, atrial premature contraction (APC), ventricular premature contraction (VPC), left bundle branch block (LBBB), and right bundle branch block (RBBB).

Specifically, the first estimation unit 210 according to one embodiment of the invention may use the first artificial neural network to estimate which of the classes representing the first type of arrhythmia at least one beat segment included in the first section of the ECG signal corresponds to, and further estimate that the class corresponding to the beat segment is a class representing a normal ECG, if the beat segment does not correspond to any of the classes representing the first type of arrhythmia.

For example, assuming that the ECG signal is inputted to the first artificial neural network, the first estimation unit 210 may use the first artificial neural network to estimate that a fourth beat segment among five beat segments included in the first section of the ECG signal corresponds to a class representing APC, and estimate that a first beat segment, a second beat segment, a third beat segment, and a fifth beat segment among the five beat segments included in the first section of the ECG signal correspond to the class representing a normal ECG.

Meanwhile, according to one embodiment of the invention, the first artificial neural network comprises an input layer, a hidden layer, and an output layer, and may be implemented as, but is not necessarily limited to, a convolutional neural network (CNN), a recurrent neural network (RNN), or the like.

Next, the second estimation unit 220 according to one embodiment of the invention may function to estimate a class corresponding to the first section of the ECG signal, using a second artificial neural network.

Here, the second artificial neural network according to one embodiment of the invention may be an artificial neural network trained to estimate which of classes representing a second type of arrhythmia a section of the ECG signal corresponds to. According to one embodiment of the invention, the second type of arrhythmia may include arrhythmia that may be estimated from rhythm changes between consecutive beat segments, and may include, for example, atrial fibrillation (AFib), paroxysmal supraventricular tachycardia (SVT), and atrioventricular block (AV block).

Specifically, the second estimation unit 220 according to one embodiment of the invention may use the second artificial neural network to estimate which of the classes representing the second type of arrhythmia the first section of the ECG signal corresponds to, and further estimate that the class corresponding to the first section is the class representing a normal ECG, if the first section does not correspond to any of the classes representing the second type of arrhythmia.

For example, assuming that the ECG signal is inputted to the second artificial neural network, the second estimation unit 220 may use the second artificial neural network to estimate that the first section of the ECG signal corresponds to a class representing AFib, or estimate that the first section of the ECG signal corresponds to the class representing a normal ECG.

According to one embodiment of the invention, the second artificial neural network may be configured in parallel with the first artificial neural network, and the same ECG signal may be inputted to the first artificial neural network and the second artificial neural network configured in parallel. That is, with respect to the same ECG signal, the first artificial neural network may estimate which of the classes representing the first type of arrhythmia the beat segment included in the first section of the ECG signal corresponds to, and the second artificial neural network may estimate which of the classes representing the second type of arrhythmia the first section of the ECG signal corresponds to. According to one embodiment of the invention, like the first artificial neural network, the second artificial neural network comprises an input layer, a hidden layer, and an output layer, and may be implemented as, but is not necessarily limited to, a convolutional neural network (CNN), a recurrent neural network (RNN), or the like.

Next, the verification unit 230 according to one embodiment of the invention may function to mutually verify the estimated class corresponding to the beat segment included in the first section of the ECG signal and the estimated class corresponding to the first section of the ECG signal.

According to one embodiment of the invention, there may be a case where the estimated class corresponding to the beat segment included in the first section of the ECG signal and the estimated class corresponding to the first section of the ECG signal are incompatible with each other. For example, there may be a case where the class corresponding to the first section of the ECG signal is estimated to be the class representing AFib and the class corresponding to the beat segment included in the first section of the ECG signal is estimated to be the class representing APC, even though APC cannot be present in the section of the ECG signal in which AFib occurs. Like the above case, the class estimation for the first section of the ECG signal or the beat segment included in the first section of the ECG signal may be incorrect, and the present invention may address such errors through a mutual verification process.

Specifically, the verification unit 230 according to one embodiment of the invention may mutually verify the estimated class corresponding to the beat segment included in the first section of the ECG signal and the estimated class corresponding to the first section of the ECG signal, and may correct one of the estimated class corresponding to the beat segment included in the first section of the ECG signal and the estimated class corresponding to the first section of the ECG signal on the basis of the other, if it is determined from a result of the verification that the class estimation for either the beat segment included in the first section of the ECG signal or the first section of the ECG signal is incorrect (i.e., the estimated class corresponding to the beat segment included in the first section of the ECG signal and the estimated class corresponding to the first section of the ECG signal are incompatible with each other).

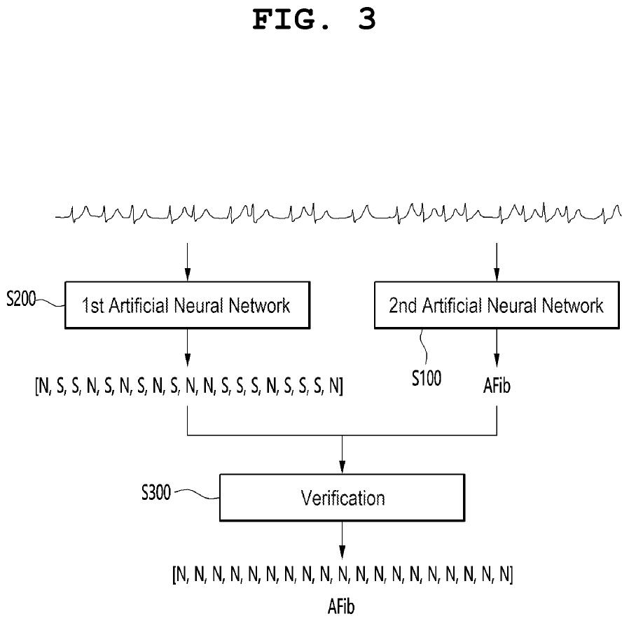

For example, as shown in FIG. 3, assuming that the second artificial neural network estimates the class corresponding to the first section of the ECG signal is the class representing AFib (S100), and that the first artificial neural network estimates each of the classes corresponding to 11 beat segments among 19 beat segments included in the first section of the ECG signal is the class representing APC (labeled “S”) and each of the classes corresponding to 8 beat segments among the 19 beat segments is the class representing a normal ECG (labeled “N”) (S200), the verification unit 230 may correct the estimated class corresponding to the 11 beat segments (i.e., the class representing APC) to the class representing a normal ECG (i.e., S->N), on the basis of the estimated class corresponding to the first section of the ECG signal (i.e., the class representing AFib) (S300).

Next, the communication unit 240 according to one embodiment of the invention may function to enable data transmission/reception from/to the first estimation unit 210, the second estimation unit 220, and the verification unit 230.

Lastly, the control unit 250 according to one embodiment of the invention may function to control data flow among of the first estimation unit 210, the second estimation unit 220, the verification unit 230, and the communication unit 240. That is, the control unit 250 according to the invention may control data flow into/out of the arrhythmia estimation system 200 or data flow among the respective components of the arrhythmia estimation system 200, such that the first estimation unit 210, the second estimation unit 220, the verification unit 230, and the communication unit 240 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for estimating arrhythmia using a composite artificial neural network, comprising the steps of:
estimating a class corresponding to a beat segment included in a first section of an electrocardiogram (ECG) signal, using a first artificial neural network;
estimating a class corresponding to the first section of the ECG signal, using a second artificial neural network; and
mutually verifying the estimated class corresponding to the beat segment included in the first section of the ECG signal and the estimated class corresponding to the first section of the ECG signal.

2. The method of claim 1, wherein the first artificial neural network and the second artificial neural network are configured in parallel, and the same ECG signal is inputted to the first artificial neural network and the second artificial neural network.

3. The method of claim 1, wherein the first artificial neural network is capable of estimating which of classes representing a first type of arrhythmia the beat segment included in the first section of the ECG signal corresponds to, and
wherein the first type of arrhythmia includes arrhythmia capable of being estimated on a beat segment basis.

4. The method of claim 1, wherein the second artificial neural network is capable of estimating which of classes representing a second type of arrhythmia the first section of the ECG signal corresponds to, and
wherein the second type of arrhythmia includes arrhythmia capable of being estimated from rhythm changes between consecutive beat segments.

5. The method of claim 1, wherein in the verifying step, one of the estimated class corresponding to the beat segment included in the first section of the ECG signal and the estimated class corresponding to the first section of the ECG signal is corrected on the basis of the other, in response to the estimated class corresponding to the beat segment included in the first section of the ECG signal and the estimated class corresponding to the first section of the ECG signal being incompatible with each other.

6. A non-transitory computer-readable recording medium having stored thereon a computer program for executing the method of claim 1.

7. A system for estimating arrhythmia using a composite artificial neural network, comprising:
a first estimation unit configured to estimate a class corresponding to a beat segment included in a first section of an electrocardiogram (ECG) signal, using a first artificial neural network;
a second estimation unit configured to estimate a class corresponding to the first section of the ECG signal, using a second artificial neural network; and
a verification unit configured to mutually verify the estimated class corresponding to the beat segment included in the first section of the ECG signal and the estimated class corresponding to the first section of the ECG signal.

8. The system of claim 7, wherein the first artificial neural network and the second artificial neural network are configured in parallel, and the same ECG signal is inputted to the first artificial neural network and the second artificial neural network.

9. The system of claim 7, wherein the first artificial neural network is capable of estimating which of classes representing a first type of arrhythmia the beat segment included in the first section of the ECG signal corresponds to, and
wherein the first type of arrhythmia includes arrhythmia capable of being estimated on a beat segment basis.

10. The system of claim 7, wherein the second artificial neural network is capable of estimating which of classes representing a second type of arrhythmia the first section of the ECG signal corresponds to, and
wherein the second type of arrhythmia includes arrhythmia capable of being estimated from rhythm changes between consecutive beat segments.

11. The system of claim 7, wherein the verification unit is configured to correct one of the estimated class corresponding to the beat segment included in the first section of the ECG signal and the estimated class corresponding to the first section of the ECG signal on the basis of the other, in response to the estimated class corresponding to the beat segment included in the first section of the ECG signal and the estimated class corresponding to the first section of the ECG signal being incompatible with each other.
